(19) United States
(12) Patent Application Publication
(10) Pub. No.: US 2011/0093026 A1
(43) Pub. Date: Apr. 21, 2011

(54) METHOD AND APPARATUS FOR CARDIORENAL ELECTRICAL STIMULATION

(76) Inventors: Ramesh Wariar, Blaine, MN (US); Jeffrey E. Stahmann, Ramsey, MN (US); Roger Hastings, Maple Grove, MN (US); Stephen Ruble, Lino Lakes, MN (US); Ronald W. Heil, Roseville, MN (US); Stephen J. Hahn, Shoreview, MN (US); Arjun Sharma, St. Paul, MN (US)

(21) Appl. No.: 12/892,694

(22) Filed: Sep. 28, 2010

Related U.S. Application Data
(60) Provisional application No. 61/252,809, filed on Oct. 19, 2009.

(51) Int. Cl. A61N 1/00

(52) U.S. Cl. 607/3

(57) ABSTRACT

An implantable cardiorenal stimulator delivers cardiorenal stimulation in response to detection of decompensation associated with heart failure. The cardiorenal stimulation includes delivering renal stimulation pulses to promote diuresis and/or natriuresis and delivering cardiac stimulation pulses to enhance the diuretic and/or natriuretic effects of the renal stimulation pulses.

### CLAIM OF PRIORITY

This application claims the benefit of provisional U.S. patent application Ser. No. 61/252,809, filed on Oct. 19, 2009, which is hereby incorporated by reference in its entirety.

### TECHNICAL FIELD

This document relates generally to implantable medical devices and particularly to an implantable cardiorenal stimulation system providing for electrical stimulation modulating cardiac and renal functions.

### BACKGROUND

The heart is the center of a person's circulatory system. It includes an electromechanical system performing two major pumping functions. The left side of the heart draws oxygenated blood from the lungs and pumps it to the organs of the body to supply their metabolic needs for oxygen. The right side of the heart draws deoxygenated blood from the body organs and pumps it to the lungs where the blood gets oxygenated. These pumping functions result from contractions of the myocardium (cardiac muscles). In a normal heart, the sinoatrial (SA) node, the heart's natural pacemaker, generates electrical impulses, called action potentials, that propagate through an electrical conduction system to various regions of the heart and excite the myocardial tissues of these regions. Coordinated delays in the propagations of the action potentials in a normal electrical conduction system cause the various portions of the heart to contract in synchrony and result in efficient pumping function.

A blocked or otherwise damaged electrical conduction system causes irregular contractions of the myocardium, a condition generally known as arrhythmia. Arrhythmia reduces the heart's pumping efficiency and hence diminishes the blood flow to the body. A deteriorated myocardium has decreased contractility, also resulting in diminished blood flow. A heart failure patient usually suffers from both a damaged electrical conduction system and a deteriorated myocardium. The diminished blood flow results in insufficient blood supply to various body organs, preventing them from functioning properly and causing various symptoms. For example, in a patient suffering from acute worsening of heart failure, an insufficient blood supply to the kidneys results in avid salt and water retention and edema in the lungs and peripheral parts of the body, a condition referred to as decompensation. Acute decompensated heart failure is a significant cause for hospitalization. Reportedly, about 25-45% of patients with compensated heart failure exhibit combined cardiac and renal dysfunction, known as cardiorenal syndrome, which is a strong risk factor for morbidity and mortality. Because acute decompensated heart failure progresses rapidly after onset, quick treatment is required upon early indications. Thus, there is a need for an efficient method and system for prompt treatment of decompensation in a heart failure patient.

### SUMMARY

An implantable cardiorenal stimulator controls delivery of cardiorenal stimulation using a detected level of water retention in a patient's body. The cardiorenal stimulation includes delivering renal electrical stimulation pulses to promote diuresis and/or natriuresis and delivering cardiac electrical stimulation pulses to enhance the diuretic and/or natriuretic effects of the renal electrical stimulation pulses.

In one embodiment, an implantable cardiorenal stimulator includes a sensing circuit, a decompensation detector, a cardiac stimulation circuit, a renal stimulation circuit, and a stimulation control circuit. The sensing circuit senses one or more physiological signals. The decompensation detector detects a level of water retention in the patient's body using the one or more physiological signals and produces a decompensation signal indicating the detected level of water retention. The cardiac stimulation circuit delivers cardiac stimulation pulses modulating cardiovascular functions. The renal stimulation circuit delivers renal stimulation pulses modulating renal functions. The stimulation control circuit controls the delivery of the cardiac stimulation pulses and the renal stimulation pulses according to a cardiorenal stimulation mode using the decompensation signal.

In one embodiment, a method for operating an implantable cardiorenal stimulator is provided. One or more physiological signals are sensed using the implantable cardiorenal stimulator. A decompensation signal indicating a level of water retention in the patient is produced using the one or more physiological signals. Delivery of cardiac stimulation pulses modulating cardiovascular functions and renal stimulation pulses modulating renal functions is controlled according to a cardiorenal stimulation mode using the decompensation signal. The cardiac stimulation pulses and the renal stimulation pulses are delivered from the implantable cardiorenal stimulator.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of a cardiorenal stimulation system and portions of an environment in which the system operates.

FIG. 2 is a block diagram illustrating an embodiment of a circuit of an implantable cardiorenal stimulator.

FIG. 3 is an illustration of an embodiment of a distal end portion of a renal stimulation lead.

FIG. 4 is an illustration of another embodiment of the distal end portion of the renal stimulation lead.

FIG. 5 is an illustration of another embodiment of the distal end portion of the renal stimulation lead and a pulse delivery stent.

FIG. 6 is an illustration of an embodiment of an anchor structure of the renal stimulation lead.

FIG. 7 is an illustration of another embodiment of the anchor structure of the renal stimulation lead.

FIG. 8 is an illustration of an embodiment of a renal stimulation lead with multiple anchoring devices.

FIG. 9 is a flow chart illustrating an embodiment of a method for cardiorenal stimulation.

### DETAILED DESCRIPTION

In the following detailed description, reference is made to the accompanying drawings which form a part hereof; and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their legal equivalents.

It should be noted that references to “an”, “one”, or “various” embodiments in this disclosure are not necessarily to the same embodiment, and such references contemplate more than one embodiment.

This document discusses, among other things, a system including an implantable cardiorenal stimulator and an implantable lead system for cardiorenal electrical stimulation. The system detects physiological changes indicative of worsening heart failure in a patient and provides prompt intervention to treat the patient, with the potential of avoiding hospitalization. In various embodiments, the worsening heart failure includes occurrence of acute decompensated heart failure, with symptoms of decompensation. The cardiorenal electrical stimulation reduces or eliminates use of diuretic drugs and hence their undesirable side effects such as hypotension, electrolyte disturbances, arrhythmia, and neurohormonal activation. In various embodiments, the cardiorenal electrical stimulation includes renal electrical stimulation (referred to as renal stimulation hereinafter) and cardiac electrical stimulation (referred to cardiac stimulation hereinafter). The renal stimulation includes delivery of electrical stimulation pulses to the kidneys and/or nerves modulating renal functions to promote diuresis and/or natriuresis. The cardiac stimulation includes delivery of electrical stimulation pulses to the heart and/or nerves modulating cardiovascular functions for enhancing the diuretic and/or natriuretic effects of the rental stimulation.

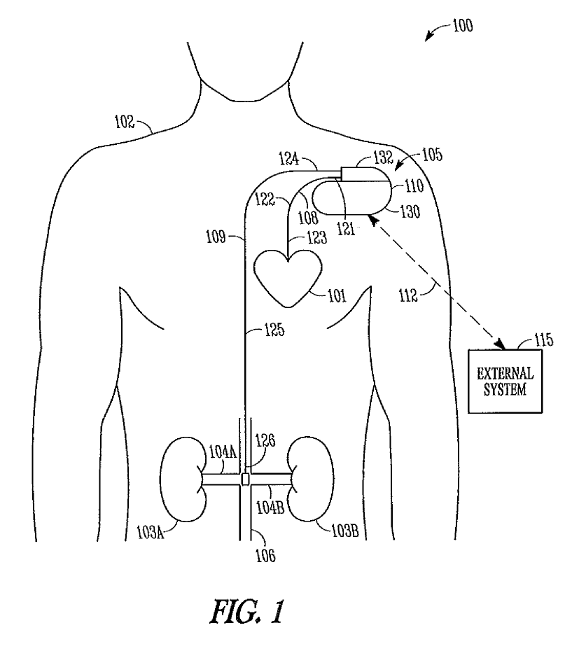

FIG. 1 is an illustration of an embodiment of a cardiorenal stimulation system 100 and portions of an environment in which system 100 operates. System 100 includes an implantable system 105, an external system 115, and a telemetry link 112 providing for communication between implantable system 105 and external system 115.

Implantable system 105 includes, among other things, implantable cardiorenal stimulator 110 and an implantable lead system including a cardiac stimulation lead 108 and a renal stimulation lead 109. In various embodiments, implantable cardiorenal stimulator 110 provides for the cardiorenal stimulation as well as other therapies such as cardioversion/defibrillation, neurostimulation, drug therapy and biological therapy. Cardiac stimulation lead 108 represents one or more implantable cardiac stimulation leads. Renal stimulation lead 109 represents one or more implantable renal stimulation leads. While FIG. 1 shows one cardiac stimulation lead and one renal stimulation lead for purposes of illustration and discussion, the implantable lead system includes any number of cardiac and renal stimulation leads, depending on, for example, the number of sites to be stimulated and accessibility considerations.

In the illustrated embodiment, implantable cardiorenal stimulator 110 is implanted in a body 102. Implantable cardiorenal stimulator 110 includes a hermetically sealed implantable housing 130 and a header 132 including a lead connector attached to housing 130. Housing 130 encapsulates electronic circuitry that performs sensing and therapeutic functions including cardiorenal stimulation. Header 132 provides for mechanical and electrical connections between leads 108 and 109 and implantable cardiorenal stimulator 110. In various embodiments, implantable cardiorenal stimulator 110 detects decompensation associated with heart failure using one or more sensed physiological signals and delivers cardiac stimulation pulses modulating cardiovascular functions and renal stimulation pulses modulating renal functions according to a cardiorenal stimulation mode in response to a detection of the decompensation.

Lead 108 provides electrical connections between implantable cardiorenal stimulator 110 and a heart 101 and/or nerves modulating cardiovascular functions. Lead 108 includes a proximal end portion 121 configured to be connected to header 132, a distal end portion 123 including one or more electrodes for delivering cardiac stimulation pulses and/or sensing one or more physiological signals, and an elongate lead body 122 coupling between proximal end portion 121 and distal end portion 123. In various embodiments, lead 108 represents one or more implantable transvenous leads for sensing physiological signals and delivering pacing pulses, cardioversion/defibrillation shocks, neurostimulation, pharmaceutical agents, biological agents, and/or other types of energy or substance for treating cardiac disorders. In various embodiments, the one or more electrodes of lead 108 are placed in a heart 101 or other portions of body 102 for sensing physiological signals and delivering pacing pulses, cardioversion/defibrillation shocks, neurostimulation, pharmaceutical agents, biological agents, and/or other types of energy or substance for treating cardiac disorders. In one embodiment, lead system 108 includes one or more pacing-sensing leads each including at least one electrode placed in or on heart 101 for sensing one or more electrograms and/or delivering pacing pulses. In a specific embodiment, lead system 108 allows pacing pulses to be delivered to multiple atrial and ventricular sites. In various embodiments, distal end portion 123, which includes the one or more pacing electrodes, is placed in the right atrium (RA) of heart 101 for baroreceptor pacing, atrial stretch receptor pacing, and/or RA pacing (for RA contraction), in the right ventricle (RV) of heart 101 for RV pacing (for RV contraction), and/or in the coronary sinus or vein over the left ventricle (LV) of heart 101 for LV pacing (for LV contraction).

Lead 109 provides electrical connections between implantable cardiorenal stimulator 110 and kidneys 103A-B and/or nerves modulating renal functions. Lead 109 includes a proximal end portion 124 configured to be connected to header 132, a distal end portion 126 including one or more electrodes for delivering renal stimulation pulses and/or sensing one or more physiological signals, and an elongate lead body 125 coupling between proximal end portion 124 and distal end portion 126. In various embodiments, lead 109 represents one or more implantable transvenous leads for sensing physiological signals and delivering renal stimulation pulses, pharmaceutical agents, biological agents, and/or other types of energy or substance for treating renal disorders. In various embodiments, distal end portion 126, which includes the one or more electrodes, is placed on kidneys 103A-B, renal veins 104A-B, an inferior vena cava (IVC) 106, or other portions of body 102 to sense physiological signals and deliver renal stimulation pulses, pharmaceutical agents, biological agents, and/or other types of energy or substance for treating renal disorders. In various embodiments, distal end portion 126 is placed in a location in body 102 that allows for delivering renal stimulation pulse to block nerve traffic in renal nerves or interconnected nerves/ganglia within the plexus.

In various embodiments, system 100 allows for bipolar cardiac stimulation using a pair of electrodes on distal end portion 123 of lead 108 and/or unipolar cardiac stimulation using an electrode on distal end portion 123 and another electrode on implantable cardiorenal stimulator 110. In various embodiments, system 100 allows for bipolar renal stimulation using a pair of electrodes on distal end portion 126 of lead 109 and/or unipolar renal stimulation using an electrode on distal end portion 126 and another electrode on implantable cardiorenal stimulator 110. In one embodiment, a portion of implantable housing 130 functions as the electrode on implantable cardiorenal stimulator 110.

External system 115 allows a user such as a physician or other caregiver or the patient to control the operation of implantable cardiorenal stimulator 110 and obtain information acquired by implantable cardiorenal stimulator 110. In one embodiment, external system 115 includes a programmer communicating with implantable cardiorenal stimulator 110 bi-directionally via telemetry link 112. In another embodiment, external system 115 is a patient management system including an external device communicating with a remote device through a telecommunication network. The external device is within the vicinity of implantable cardiorenal stimulator 110 and communicates with implantable cardiorenal stimulator 110 bi-directionally via telemetry link 112. The remote device allows the user to monitor and treat a patient from a distant location.

Telemetry link 112 provides for data transmission from implantable cardiorenal stimulator 110 to external system 115. This includes, for example, transmitting real-time physiological data acquired by implantable cardiorenal stimulator 110, extracting physiological data acquired by and stored in implantable cardiorenal stimulator 110, extracting therapy history data stored in implantable cardiorenal stimulator 110, and extracting data indicating an operational status of implantable cardiorenal stimulator 110 (e.g., battery status and lead impedance). Telemetry link 112 also provides for data transmission from external system 115 to implantable cardiorenal stimulator 110. This includes, for example, programming implantable cardiorenal stimulator 110 to acquire physiological data, programming implantable cardiorenal stimulator 110 to perform at least one self-diagnostic test (such as for a device operational status), and programming implantable cardiorenal stimulator 110 to deliver one or more therapies.

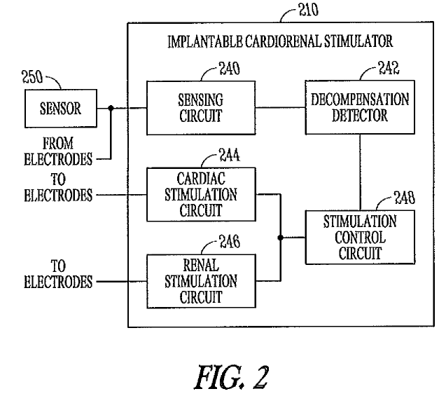

FIG. 2 is a block diagram illustrating an embodiment of a circuit of an implantable cardiorenal stimulator 210. Implantable cardiorenal stimulator 210 is an embodiment of implantable cardiorenal stimulator 110 and includes a sensing circuit 240, a decompensation detector 242, a cardiac stimulation circuit 244, a renal stimulation circuit 246, and a stimulation control circuit 248. In various embodiments, implantable cardiorenal stimulator 210 includes and/or connects to a sensor 250. Sensor 250 represents one or more physiological sensors, including one or more sensors encapsulated in housing 130 and/or one or more sensors external to housing 130 but communicatively connected to the circuitry encapsulated in housing 130.

Sensing circuit 240 senses one or more physiological signals via one or more electrodes on lead 108, lead 109, and/or sensor 250. The one or more physiological signals include one or more signals indicative of water and/or salt retention in body 102. Examples of such signals includes signals indicative of body tissue volume, blood volume, and effects of abnormal tissue or blood volumes, as further discussed below. Decompensation detector 242 detects a level of water retention in body 102 using the one or more physiological signals and produces a decompensation signal indicative of the detected level of water retention. In one embodiment, decompensation detector 242 detects occurrence of decompensation associated with heart failure using the one or more physiological signals and one or more specified thresholds. In one embodiment, the one or more thresholds are each specified according to a need to deliver therapy indicated by one of the one or more physiological signals. In one embodiment, the decompensation signal includes a decompensation alert signal indicative of an occurrence of decompensation. In various embodiments, the decompensation alert signal indicates onset and cessation of the detected decompensation and/or a status or degree of the detected decompensation. For example, the decompensation alert signal includes a parameter value that quantitatively indicates the status or degree of the detected decompensation.

In one embodiment, decompensation detector 242 detects conditions indicative of decompensation that occur during acute decompensated heart failure. In another embodiment, decompensation detector 242 detects conditions that may lead to acute decompensated heart failure or conditions indicative of recovery from acute decompensated heart failure. Heart failure results in diminished blood flow from the heart as measured by cardiac output or stroke volume. Cardiac output is the amount of blood pumped by the heart during a unit period of time. Stroke volume is the amount of blood pumped during each contraction or stroke. Decompensated heart failure occurs when the heart becomes significantly weakened such that the body's compensatory mechanisms cannot restore a normal cardiac output/stroke volume. One principal consequence of the decompensated heart failure is that the heart fails to provide the kidneys with sufficient blood to support normal renal functions. As a result, a patient suffering decompensated heart failure progressively develops increased neurohormonal activation, retention of salt and water, and ultimately pulmonary and peripheral edema, a process referred to as decompensation.

In one embodiment, sensor 250 includes an implantable impedance sensor to measure pulmonary impedance, or impedance of a portion of the thoracic cavity. In another embodiment, sensor 250 includes an implantable impedance sensor to measure blood impedance indicative of hemodilution resulting from water retention. Decompensation detector 242 produces the decompensation alert signal when the impedance is out of its normal range. For example, pulmonary edema, i.e., fluid retention in the lungs resulting from the decreased cardiac output, increases the pulmonary or thoracic impedance. In one specific embodiment, decompensation detector 242 produces the decompensation alert signal when the pulmonary or thoracic impedance exceeds a specified threshold impedance. In one embodiment, the impedance sensor is a respiratory sensor that senses the patient's minute ventilation. An example of an impedance sensor sensing minute ventilation is discussed in U.S. Pat. No. 6,459,929, “IMPLANTABLE CARDIAC RHYTHM MANAGEMENT DEVICE FOR ASSESSING STATUS OF CHF PATIENTS,” assigned to Cardiac Pacemakers, Inc., which is incorporated herein by reference in its entirety.

In one embodiment, sensor 250 includes a pressure sensor. Acute decompensated heart causes pressures in various portions of the cardiovascular system to deviate from their normal ranges. Decompensation detector 242 produces the decompensation alert signal when a pressure is outside of its normal range. Examples of the pressure sensor include a central venous pressure (CVP) sensor, left atrial (LA) pressure sensor, a left ventricular (LV) pressure sensor, an artery pressure sensor, a pulmonary artery pressure sensor, and an intra-abdominal pressure sensor. In various embodiments, one or more of such pressure sensors are incorporated into one or more of lead 108, lead 109, and housing 130. Pulmonary edema results in elevated LA and pulmonary arterial pressures. A deteriorated LV results in decreased LV and arterial pressures. In various embodiments, decompensation detector 242 produces the decompensation alert signal when the LA pressure exceeds a specified threshold LA pressure level, when the pulmonary arterial pressure exceeds a predetermined threshold pulmonary arterial pressure level, when the LV pressure falls below a predetermined threshold LV pressure level, and/or when the arterial pressure falls below a predetermined threshold LV pressure level. In other embodiments, decompensation detector 242 derives a parameter from one of these pressures, such as a rate of change of a pressure, and produces the decompensation alert signal when the parameter deviates from its normal range. In one embodiment, the LV pressure sensor senses the LV pressure indirectly, by sensing a signal having known or predictable relationships with the LV pressure during all or a portion of the cardiac cycle. Examples of such a signal include an LA pressure and a coronary vein pressure. One specific example of measuring the LV pressure using a coronary vein pressure sensor is discussed in U.S. patent application Ser. No. 10/038,936, “METHOD AND APPARATUS FOR MEASURING LEFT VENTRICULAR PRESSURE,” filed on Jan. 4, 2002, assigned to Cardiac Pacemakers, Inc., which is hereby incorporated by reference in its entirety.

In one embodiment, sensor 250 includes a cardiac output or stroke volume sensor. Examples of stroke volume sensing are discussed in U.S. Pat. No. 4,686,987, “BIOMEDICAL METHOD AND APPARATUS FOR CONTROLLING THE ADMINISTRATION OF THERAPY TO A PATIENT IN RESPONSE TO CHANGES IN PHYSIOLOGIC DEMAND,” and U.S. Pat. No. 5,284,136, “DUAL INDIFFERENT ELECTRODE PACEMAKER,” both assigned to Cardiac Pacemakers, Inc., which are incorporated herein by reference in their entirety. Decompensation detector 242 produces the decompensation alert signal when the stroke volume falls below a specified threshold level.

In one embodiment, sensor 250 includes a neural activity sensor to detect activities of the sympathetic nerve and/or the parasympathetic nerve. A significant decrease in cardiac output immediately stimulates sympathetic activities, as the autonomic nervous system attempts to compensate for deteriorated cardiac function. Sympathetic activities sustain even when the compensation fails to restore the normal cardiac output. In one specific embodiment, the neural activity sensor includes a neurohormone sensor to sense a hormone level of the sympathetic nerve and/or the parasympathetic nerve. Decompensation detector 242 produces the decompensation alert signal when the hormone level exceeds a specified threshold level. In another specific embodiment, the neural activity sensor includes an action potential recorder to sense the electrical activities in the sympathetic nerve and/or the parasympathetic nerve. Decompensation detector 242 produces the decompensation alert signal when the frequency of the electrical activities in the sympathetic nerve exceeds a predetermined threshold level. Examples of direct and indirect neural activity sensing are discussed in U.S. Pat. No. 5,042,497, “ARRHYTHMIA PREDICTION AND PREVENTION FOR IMPLANTED DEVICES,” assigned to Cardiac Pacemakers, Inc., which is hereby incorporated by reference in its entirety. In one embodiment, decompensation detector 242 includes a heart rate variability detector. Patients suffering acute decompensated heart failure exhibit abnormally low heart rate variability. An example of detecting the heart rate variability is discussed in U.S. Pat. No. 5,603,331, “DATA LOGGING SYSTEM FOR IMPLANTABLE CARDIAC DEVICE,” assigned to Cardiac Pacemakers, Inc., which is incorporated herein by reference in their entirety. Decompensation detector 242 produces the decompensation alert signal when the heart rate variability falls below a specified threshold level.

In one embodiment, sensor 250 includes a renal function sensor. Acute decompensated heart failure results in peripheral edema primarily because of fluid retention by the kidneys that follows the reduction in cardiac output. The fluid retention is associated with reduced renal output, decreased glomerular filtration, and formation of angiotensin. Thus, in one specific embodiment, the renal function sensor includes a renal output sensor to sense a signal indicative of the renal output. In one embodiment, decompensation detector 242 produces the decompensation alert signal when the CVP exceeds a threshold CVP pressure (such as 15-20 mm Hg) or when intra-abdominal pressure exceeds a threshold intra-abdominal pressure. In another embodiment, decompensation detector 242 produces the decompensation alert signal when the sensed renal output falls below a predetermined threshold. In another specific embodiment, the renal function sensor includes a filtration rate sensor to sense a signal indicative of the glomerular filtration rate. Decompensation detector 242 produces the decompensation alert signal when the sensed glomerular filtration rate falls below a specified threshold. In yet another specific embodiment, the renal function sensor includes a chemical sensor to sense a signal indicative of increased rennin or angiotensin levels. Decompensation detector 242 produces the decompensation alert signal when the sensed rennin or angiotensin level exceeds a specified threshold level.

In one embodiment, sensor 250 includes an acoustic sensor being a heart sound sensor and/or a respiratory sound sensor. Acute decompensated heart failure causes abnormal cardiac and pulmonary activity patterns and hence, deviation of heart sounds and respiratory sounds from their normal ranges of pattern and/or amplitude. Decompensation detector 242 produces the decompensation alert signal when the heart sound or respiratory sound is out of its normal range. For example, detection of the third heard sound (S3) is known to indicate heart failure. In one specific embodiment, decompensation detector 242 produces the decompensation alert signal when the S3 amplitude exceeds a predetermined threshold level.

Embodiments of sensor 250 and decompensation detector 242 are discussed in this document by way of example, but not by way of limitation. Other methods and sensors for directly or indirectly detecting decompensation, as known to those skilled in the art, are useable as sensor 250 and decompensation detector 242.

Cardiac stimulation circuit 244 delivers cardiac stimulation pulses modulating cardiovascular functions. In one embodiment, cardiac stimulation circuit 244 delivers cardiac pacing pulses to the heart via lead 108 having one or more electrodes placed in or on the heart. In another embodiment, cardiac stimulation circuit 244 delivers neural pacing pulses to deliver neural pacing pulses to a nervous system modulating cardiac functions via lead 108 having one or more electrodes placed on or adjacent baroreceptors and/or other components of the autonomic neural system.

Renal stimulation circuit 244 delivers renal stimulation pulses modulating renal functions. In one embodiment, renal stimulation circuit 244 delivers renal stimulation pulses to the kidneys via lead 109 having one or more electrodes placed on or adjacent to the kidneys. In another embodiment, renal stimulation circuit 244 delivers renal stimulation pulses to renal nerves via lead 109 having one or more electrodes placed on or adjacent one or more of nerves, ganglia, and plexuses that innervate the kidney, including the aorticorenal ganglion, renal ganglia, celiac plexus, intermesenteric plexus, paravertebral sympathetic chain, prevertebral ganglia, splanchnic nerve, renal nerves, and vagus nerve.

Stimulation control circuit 248 controls the delivery of the cardiac stimulation pulses and the renal stimulation pulses according to a cardiorenal stimulation mode using renal stimulation parameters and cardiac stimulation parameters. In various embodiments, stimulation control circuit 248 temporally coordinates delivery of the cardiac stimulation pulses and the renal stimulation pulses according to the cardiorenal stimulation mode. In various embodiments, stimulation control circuit 248 controls the delivery of the cardiac stimulation pulses and the delivery of the renal stimulation pulses using the decompensation signal produced by decompensation detector 242. In one embodiment, stimulation control circuit 248 provides closed-loop control of the delivery of the cardiac stimulation pulses and the delivery of the renal stimulation pulses using the decompensation signal as a feedback input. In one embodiment, stimulation control circuit 248 controls the delivery of the cardiac stimulation pulses and the delivery of the renal stimulation pulses using the decompensation signal and one or more additional control signals. In various embodiments, stimulation control circuit 248 uses the one or more additional control signals to control timing and/or intensity of the cardiac stimulation pulses and the renal stimulation pulses. Examples of such control signals include signals indicative of drug therapy received by the patient and time of the day.

In one embodiment, stimulation control circuit 248 initiates the delivery of the cardiac stimulation pulses and the renal stimulation pulses according to the cardiorenal stimulation mode in response to the onset of the decompensation as indicated by the decompensation signal. In one embodiment, stimulation control circuit 248 stops the delivery of the cardiac stimulation pulses and the renal stimulation pulses according to the cardiorenal stimulation mode in response to the cessation of the decompensation as indicated by the decompensation signal.

In one embodiment, stimulation control circuit 248 controls the delivery of the renal stimulation pulses using renal stimulation parameters selected for increasing diuresis and natriuresis by modifying renal adrenergic drive and/or renal cholinergic drive. In one embodiment, the renal stimulation parameters are selected for partially or completely blocking renal adrenergic drive. In another embodiment, the renal stimulation parameters are selected for enhancing renal cholinergic drive, in place of or in addition to partially or completely blocking renal adrenergic drive. The delivery of the renal stimulation pulses is controlled to increase renal perfusion, reduce renin secretion and renin-angiotensin-aldosterone system (RAAS) activation, or alter renal filtration or renal reabsorption (such as by reducing sodium reabsorption in the proximal tubule), thereby promoting diuresis and natriuresis. In one embodiment, stimulation control circuit 248 controls the delivery of the renal stimulation pulses to override the renal nerve traffic using a neurostimulation frequency substantially higher than the frequency of the intrinsic action potential impulses in the renal nerves. In another embodiment, stimulation control circuit 248 controls the delivery of the renal stimulation pulses to hyperpolarize the renal nerves.

In one embodiment, stimulation control circuit 248 controls the delivery of the cardiac stimulation pulses using cardiac stimulation parameters selected for enhancing one or more effects of the delivery of the renal stimulation pulses. Examples of such one or more effects include diuretic and natriuretic effects. The cardiac stimulation pulses include cardiac pacing pulses and/or neural pacing pulses. In one embodiment, stimulation control circuit 248 controls the delivery of the cardiac pacing pulses according to an anti-bradycardia pacing mode using a pacing rate that is substantially higher than the patient's intrinsic heart rate. In another embodiment, stimulation control circuit 248 controls the delivery of the cardiac pacing pulses according to a cardiac resynchronization therapy (CRT) pacing mode to improve hemodynamic performance using cardiac pacing. In one embodiment, stimulation control circuit 248 controls delivery of the neural pacing pulses for atrial stretch pacing. In one embodiment, stimulation control circuit 248 controls delivery of the neural pacing pulse for baroreceptor pacing.

In various embodiments, implantable housing 130 encapsulates at least sensing circuit 240, decompensation detector 242, cardiac stimulation circuit 244, renal stimulation circuit 246, and stimulation control circuit 248. Header 132 is electrically connected to sensing circuit 240, cardiac stimulation circuit 244, and renal stimulation circuit 246.

In various embodiments, implantable cardiorenal stimulator 210 is implemented using a combination of hardware and software. In various embodiments, each element of implantable cardiorenal stimulator 210 may be implemented using an application-specific circuit constructed to perform one or more specific functions or a general-purpose circuit programmed to perform such function(s). Such a general-purpose circuit includes, but is not limited to, a microprocessor or a portion thereof, or other programmable logic circuit or a portion thereof. In one embodiment, decompensation detector 242 and stimulation control circuit 248 are implemented as a microprocessor-based circuit programmed to perform various functions discussed in this document.

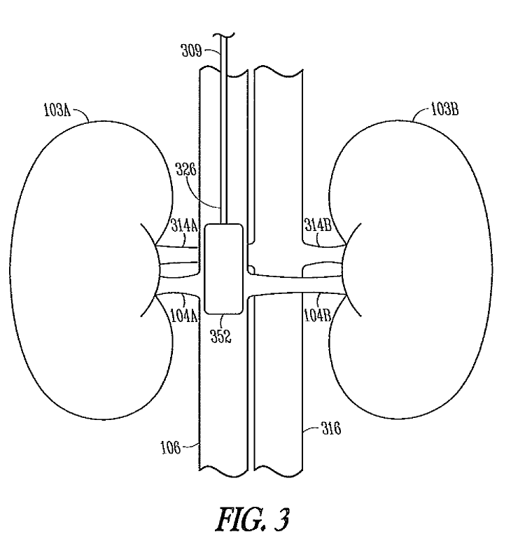

FIGS. 3-8 illustrate various embodiments of renal stimulation lead 109, showing the distal end portion in particular. In one embodiment, lead 109 is implanted using a technique that is substantially similar to the technique for implanting a cardiac pacing lead. A subclavian puncture, cephalic cutdown, or external jugular access is used to pass distal end portion 126 of lead 109 through the superior vena cava (SVC), the RA, and the IVC, such that distal end portion 126 can be placed in the IVC or one or more renal veins. FIGS. 3-5 also illustrate portions of an environment in which the distal end portion of lead 109 is deployed, showing kidneys 103A-B, renal veins 104A-B, IVC 106, renal arteries 314A-B, and an aorta 316.

FIG. 3 is an illustration of an embodiment of a distal end portion 326 of a renal stimulation lead 309. Lead 309 represents an embodiment of lead 109. Distal end portion 326 represents an embodiment of distal end portion 126 and includes an anchoring structure 352. After lead 309 is implanted, anchoring structure 352 stabilizes distal end portion 326 in IVC 106.

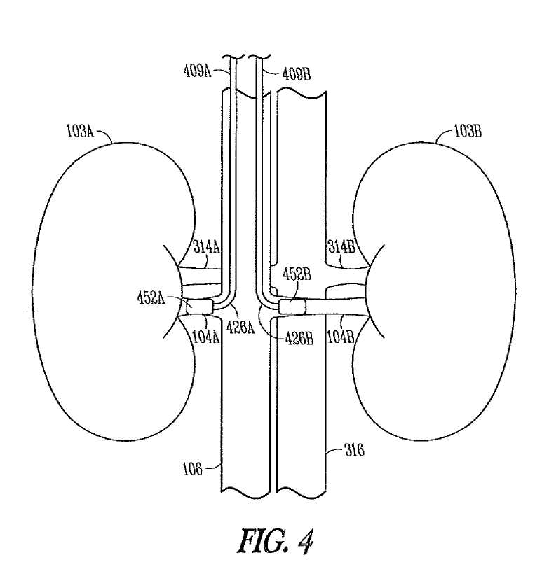

FIG. 4 is an illustration of an embodiment of a distal end portion 426A of a renal stimulation lead 409A and a distal end portion 426B of a renal stimulation lead 409B. Leads 409A-B represent another embodiment of lead 109. In one embodiment, leads 409A-B represent two separate leads. In another embodiment, leads 409A-B represent two branches of a lead that separate at the distal end portion. Distal end portions 426A-B represent another embodiment of distal end portion 126 and include anchoring structures 452A-B. After leads 409A-B are implanted, anchoring structure 452A stabilizes distal end portion 426A in renal vein 104A, and anchoring structure 452B stabilizes distal end portion 426B in renal vein 104B.

FIG. 5 is an illustration of an embodiment of a distal end portion 526 of a renal stimulation lead 509 and a pulse delivery stent 560. Lead 509 represents another embodiment of lead 109. Distal end portion 526 represents another embodiment of distal end portion 126 and includes an anchoring structure 552 and a transmitter 554. After lead 509 is implanted, anchoring structure 552 stabilizes distal end portion 526 in IVC 106. Transmitter 554 transmits power and renal stimulation pulses to pulse delivery stent 560. In various embodiments, transmitter 554 receives the power and the renal stimulation pulses from implantable cardiorenal stimulator 110, and relays the power and the renal stimulation pulses to pulse delivery stent 560 via a magnetic (inductive) or acoustic couple.

In the illustrated embodiment, pulse delivery stein 560 is placed in aorta 316, which allows electrodes to be placed in a location closer to the target renal nerves than IVC 106. Pulse delivery stent 560 includes electrodes 558A-B, a receiver 556, and a pulse delivery circuit 557. Receiver 556 receives the power and the renal stimulation pulses. In one embodiment, transmitter 554 and receiver 556 each include a coil (or antenna) to form the magnetic (inductive) couple via which the power and the renal stimulation pulses are transmitted. In one embodiment, transmitter 554 and receiver 556 each include an acoustic transducer to form the acoustic couple via which the power and the renal stimulation pulses are transmitted. Pulse delivery circuit 557 operates with the received power and delivers electrical stimulation pulses corresponding to the received renal stimulation pulses.

In various embodiments, distal end portion 526 of renal stimulation lead 509 and pulse delivery stent 560 are configured and placed with considerations on desirable stimulation target(s) and/or stability of the placement. In one embodiment, one or more leads with electrodes are connected to pulse delivery stent 560 to allow pulse delivery stent 560 to be placed in aorta 316 while the electrodes are placed in renal arteries 314A-B. In another embodiment, distal end portion 526 is placed in renal vein 104A or 104B, and pulse delivery stent 560 is placed in the adjacent renal artery 314A or 314B. In another embodiment, multiple leads or a lead with multiple distal end portions, as well as multiple pulse delivery stents, are used, such that a distal end portion is placed in each of renal veins 104A-B, and pulse delivery stents are placed in each of renal arteries 314A-B.

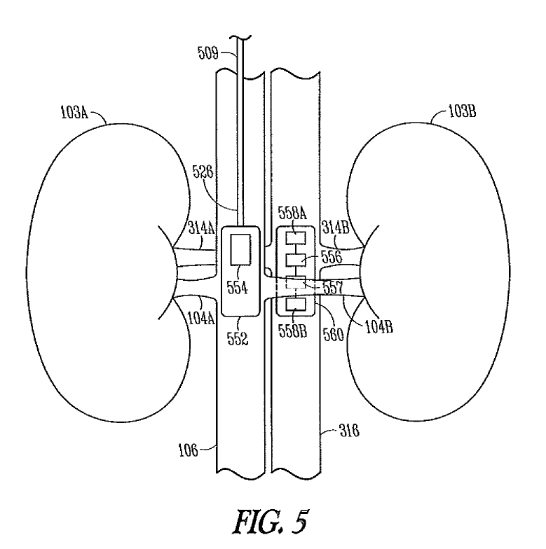

FIG. 6 is an illustration of an embodiment of an anchor structure 652 of a renal stimulation lead 609. Renal stimulation lead 609 represents an embodiment of lead 109 showing its distal end portion 626, which includes anchor structure 652. Anchor structure 652 represents an embodiment of anchor structures 352, 452A, 452B, or 552, and includes one or more spiral portions configured to be in contact of the internal wall of a blood vessel such as IVC 106 or renal vein 104A or 104B after implantation. In the illustrated embodiment, electrodes 670A-E are incorporated onto anchor structure 652 for sensing and/or delivering the renal stimulation pulses. In various embodiments, any number of electrodes are incorporated onto anchor structure 652. In one embodiment, multiple electrodes such as electrodes 670A-E on distal end portion 626 allow for electronic repositioning of electrodes in which stimulation control circuit 248 selects one or more active electrodes for sensing and/or pulse delivery purposes based on the quality of sensed signal(s), amplitude of the renal stimulation pulses required to elicit intended responses (stimulation threshold), and whether the delivery of the renal stimulation pulses results in unintended stimulation or hyperpolarization of adjacent nerves.

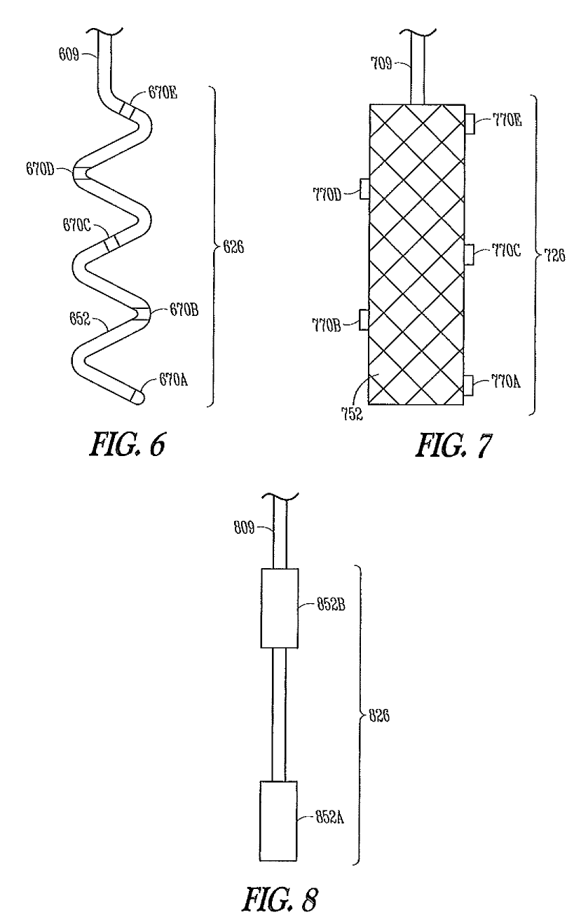

FIG. 7 is an illustration of an embodiment of an anchor structure 752 of a renal stimulation lead 709. Renal stimulation lead 709 represents another embodiment of lead 109 showing its distal end portion 726, which includes anchor structure 752. Anchor structure 752 represents another embodiment of anchor structures 352, 452A, 452B, or 552, and includes a stent configured to be in contact with the internal wall of a blood vessel such as IVC 106 or renal vein 104A or 104B after being expanded during implantation. In the illustrated embodiment, electrodes 770A-E are incorporated onto anchor structure 752 for sensing and/or delivering the renal stimulation pulses. In various embodiments, any number of electrodes are incorporated onto anchor structure 752. In one embodiment, multiple electrodes such as electrodes 770A-E on distal end portion 726 allow for electronic repositioning of electrodes in which stimulation control circuit 248 selects one or more active electrodes for sensing and/or pulse delivery purposes based on the quality of sensed signal(s), amplitude of the renal stimulation pulses required to elicit intended responses (stimulation threshold), and whether the delivery of the renal stimulation pulses results in unintended stimulation or hyperpolarization of adjacent nerves.

FIG. 8 is an illustration of an embodiment of a renal stimulation lead 809 with multiple anchoring devices. Renal stimulation lead 809 represents another embodiment of lead 109 showing its distal end portion 826. In the illustrated embodiment, distal end portion 826 includes anchoring structures 852A-B. In various embodiments, distal end portion 826 includes any number of anchoring structures to stabilize lead 809 in a blood vessel and prevent its dislodgement. In various embodiments, anchoring structures 852A-B each have the configuration of anchoring structure 652 or anchoring structure 752.

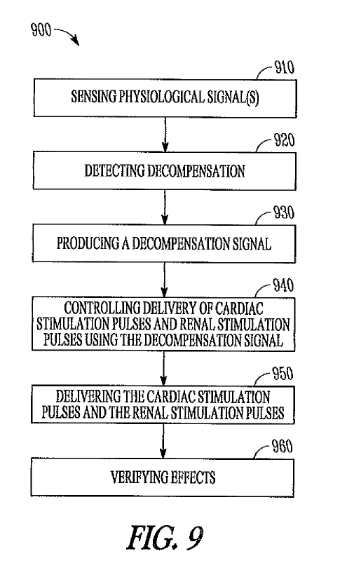

FIG. 9 is a flow chart illustrating an embodiment of a method 900 for cardiorenal stimulation. In one embodiment, method 900 is performed using system 100.

At 910, one or more physiological signals are sensed using an implantable medical device such as implantable cardiorenal stimulator 110. The one or more physiological signals indicate a level of water retention in a patient's body. Occurrence of decompensation during acute decompensated heart failure is detected using the one or more physiological signals. At 920, decompensation associated with heart failure is detected using the one or more physiological signals. At 930, a decompensation signal is produced in response to a detected decompensation. In one embodiment, the decompensation signal is indicative of an onset and cessation of the detected decompensation. In another embodiment, the decompensation signal is quantitatively indicative of a status or degree of the detected decompensation. In yet another embodiment, the decompensation signal is quantitatively indicative of a status or degree of a condition that may lead to acute decompensated heart failure or recovery from acute decompensated heart failure.

At 940, delivery of cardiac stimulation pulses modulating cardiovascular functions and renal stimulation pulses modulating renal functions is controlled according to a cardiorenal stimulation mode using the decompensation signal. In one embodiment, the delivery of the cardiac stimulation pulses and the delivery of the renal stimulation pulses are temporally coordinated such that the cardiac stimulation pulses are delivered to enhance one or more effects of the delivery of the renal stimulation pulses. In one embodiment, the delivery of the cardiac stimulation pulses and the renal stimulation pulses according to the cardiorenal stimulation mode is initiated in response to the onset of the detected decompensation and stopped in response to the cessation of the detected decompensation. In one embodiment, the delivery of the cardiac stimulation pulses and the renal stimulation pulses is controlled according to the cardiorenal stimulation mode using the decompensation signal as a feedback input in a closed-loop control system. In one embodiment, timing and intensity of the delivery of the cardiac stimulation pulses and the renal stimulation pulses is controlled using the decompensation signals and one or more additional signals.

At 950, the cardiac stimulation pulses and the renal stimulation pulses are delivered from the implantable medical device such as implantable cardiorenal stimulator 110. In various embodiments, the cardiac stimulation pulses include cardiac pacing pulses delivered to a heart and/or neural pacing pulses delivered to a nervous system modulating cardiovascular functions. The renal stimulation pulses include renal stimulation pulses delivered to one or more kidneys and/or one or more renal nerves to increase diuresis or natriuresis by blocking renal adrenergic drive and/or enhancing renal cholinergic drive. In one embodiment, the renal stimulation pulses are delivered via one or more electrodes placed in the IVC and/or renal vein(s) adjacent to renal nerve(s). In another embodiment, the renal stimulation pulses are delivered via one or more electrodes placed in an artery adjacent to renal nerve(s) and a wireless link coupling the one or more electrodes to the implantable medical device.

At 960, one or more effects of the delivery of the cardiac stimulation pulses and the renal stimulation pulses are verified by monitoring one or more signals each indicative of diuresis or natriuresis. In one embodiment, a Foley type catheter with one or more sensors is used. Examples of the one or more sensors include a flow sensor for direct diuresis measurement and a conductivity sensor for indirect measurement of sodium secretion. In another embodiment, ultrasonic or other imaging techniques are employed for measuring bladder volume before and after renal stimulation.

In one embodiment, method 900 is performed using system 100 as a chronic therapy for a heart failure patient. Sensing circuit 240 and decompensation detector 242 are chronically enabled. Cardiorenal stimulation is controlled using the compensation signal produced by decompensation detector 242 in response to each detection of decompensation. In one embodiment, cardiac stimulation parameters are adjusted as needed for enhancing diuresis and natriuresis. This includes, for example, applying CRT pacing to enhance diuresis and natriuresis by improving hemodynamic performance. The hemodynamic performance is monitored and approximately optimized by adjusting pacing parameters such as atrioventricular (AV) delay and interventricular (VV) delay. In various embodiments, the cardiac stimulation includes one or more of (1) stimulation of cardiopulmonary reflexes that improve diuresis via reduction of sympathetic activation (making renal nerve blocking more effective) or reduction of antidiuretic hormone (ADH), (2) hyperpolarization of sympathetic cardiac afferents that reduces sympathetic activation, (3) stimulation of cardiopulmonary receptors in the RA, LA, and coronary sinus that activates the cardiopulmonary stretch reflex, either during RA and LA refractory periods or using continuous stimulation of nerve endings using cardiac subthreshold stimulation currents, and (4) anti-bradycardia or CRT pacing that increases cardiac output. In one embodiment, when the patient's heart failure status is stable, the cardiorenal stimulation is applied on a periodic basis and/or based on the one or more physiological signals, to reduce dose of diuretic drug, preserve glomerular filtration rate (GFR) and prevent progression of chronic kidney disease (CKD) stage by reducing sympathetic drive to the kidneys, and/or control blood pressure.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering electrical stimulation to a living body having blood vessels, the system comprising:
a sensing circuit adapted to sense one or more physiological signals;
a decompensation detector programmed to detect a level of water retention in the living body using the one or more physiological signals and produce a decompensation signal indicative of the detected level of water retention;
a cardiac stimulation circuit adapted to deliver cardiac stimulation pulses modulating cardiovascular functions;
a renal stimulation circuit adapted to deliver renal stimulation pulses modulating renal functions;
a stimulation control circuit coupled to the decompensation detector, the renal stimulation circuit, and the cardiac stimulation circuit, the stimulation control circuit programmed to control the delivery of the cardiac stimulation pulses and the renal stimulation pulses according to a cardiorenal stimulation mode using the decompensation signal; and
an implantable housing encapsulating the sensing circuit, the decompensation detector, the cardiac stimulation circuit, the renal stimulation circuit, and the stimulation control circuit.

2. The system of claim 1, wherein the stimulation control circuit is programmed to control the delivery of the renal stimulation pulses using renal stimulation parameters selected for increasing at least one of diuresis and natriuresis by modifying at least one of renal adrenergic drive and renal cholinergic drive and control the delivery of the cardiac stimulation pulses using cardiac stimulation parameters selected for enhancing one or more effects of the delivery of the renal stimulation pulses.

3. The system of claim 2, wherein the decompensation detector is programmed to detect decompensation associated with heart failure, the decompensation signal is indicative of onset and cessation of the decompensation, and the stimulation control circuit is programmed to initiate the delivery of the cardiac stimulation pulses and the renal stimulation pulses according to the cardiorenal stimulation mode in response to the onset of the decompensation and stop the delivery of the cardiac stimulation pulses and the renal stimulation pulses according to the cardiorenal stimulation mode in response to the cessation of the decompensation.

4. The system of claim 2, wherein the stimulation control circuit is programmed to control the delivery of the cardiac stimulation pulses and the renal stimulation pulses according to the cardiorenal stimulation mode using the decompensation signal as a feedback input.

5. The system of claim 4, wherein the stimulation control circuit is programmed to control delivery of cardiac pacing pulses according to a cardiac resynchronization therapy pacing mode and adjust one or more of an atrioventricular delay and an interventricular delay using the one or more physiological signals.

6. The system of claim 1, comprising:
a header attached to the implantable housing; and
an implantable lead system adapted to be connected to the header and electrically connected to the sensing circuit, the cardiac stimulation circuit, and the renal stimulation circuit through the header, the implantable lead system including:
one or more transvenous cardiac stimulation leads; and
one or more transvenous renal stimulation leads each including:
a proximal end portion adapted to be connected to the header;
a distal end portion including one or more anchoring structures each adapted to stabilize the distal end portion in at least one of the blood vessels; and
an elongate body portion coupled between the proximal end portion and the distal end portion.

7. The system of claim 6, wherein the one or more anchoring structures each comprise a spiral portion or a stent.

8. The system of claim 7, wherein the one or more transvenous renal stimulation leads each comprise a plurality of anchoring structures.

9. The system of claim 6, wherein the one or more transvenous renal stimulation leads each comprise a plurality of electrodes incorporated into the one or more anchoring structures, and the stimulation control circuit is programmed to select one or more electrodes of the plurality of electrodes for delivering the renal stimulation pulses.

10. The system of claim 6, wherein the one or more transvenous renal stimulation leads comprise a first transvenous renal stimulation lead including a transmitter adapted to transmit the renal stimulation pulses, and comprising a stent wirelessly coupled to the transmitter, the stent including:
electrodes;
a receiver adapted to receive the renal stimulation pulses; and
a pulse delivery circuit adapted to deliver electrical pulses corresponding to the renal stimulation pulses.

11. A method for operating an implantable cardiorenal stimulator placed in a living body, the method comprising:
sensing one or more physiological signals indicative of a level of water retention in the living body using the implantable cardiorenal stimulator;
producing a decompensation signal indicative of the detected level of water retention;
controlling delivery of cardiac stimulation pulses modulating cardiovascular functions and renal stimulation pulses modulating renal functions according to a cardiorenal stimulation mode using the decompensation signal; and
delivering the cardiac stimulation pulses and the renal stimulation pulses from the implantable cardiorenal stimulator.

12. The method of claim 11, comprising detecting decompensation associated with heart failure using the sensed one or more physiological signals and one or more specified thresholds, and wherein producing the decompensation signal comprises producing a decompensation signal indicative of an onset and cessation of the detected decompensation, and controlling the delivery of the cardiac stimulation pulses and the renal stimulation pulses comprises initiating the delivery of the cardiac stimulation pulses and the renal stimulation pulses according to the cardiorenal stimulation mode in response to the onset of the detected decompensation and stopping the delivery of the cardiac stimulation pulses and the renal stimulation pulses according to the cardiorenal stimulation mode in response to the cessation of the detected decompensation.

13. The method of claim 11, wherein controlling the delivery of the cardiac stimulation pulses and the renal stimulation pulses comprises controlling the delivery of the cardiac stimulation pulses and the renal stimulation pulses according to the cardiorenal stimulation mode using the decompensation signal as a feedback input in a closed-loop control system.

14. The method of claim 11, wherein delivering the renal stimulation pulses comprises delivering the renal stimulation pulses to one or more renal nerves to increase at least one of diuresis and natriuresis by modifying at least one of renal adrenergic drive and renal cholinergic drive.

15. The method of claim 14, comprising temporally coordinating the delivery of the cardiac stimulation pulses with the delivery of the renal stimulation pulses such that the cardiac stimulation pulses are delivered to enhance one or more effects of the delivery of the renal stimulation pulses.

16. The method of claim 15, wherein delivering the cardiac stimulation pulses comprises:
delivering cardiac pacing pulses to a heart; and
delivering neural pacing pulses to a nervous system modulating the cardiovascular functions.

17. The method of claim 11, comprising delivering the renal stimulation pulses to one or more renal nerves through one or more electrodes placed in an inferior vena cava.

18. The method of claim 11, comprising delivering the renal stimulation pulses to one or more renal nerves through one or more electrodes placed in one or more renal veins.

19. The method of claim 11, comprising delivering the renal stimulation pulses to one or more renal nerves through one or more electrodes placed in an artery adjacent to a renal nerve and a wireless link coupling the one or more electrodes to the implantable cardiorenal stimulator.

20. The method of claim 11, comprising verifying one or more effects of the delivery of the cardiac stimulation pulses and the renal stimulation pulses by monitoring one or more signals each indicative of diuresis or natriuresis.
